(19) United States
(12) Patent Application Publication
(10) Pub. No.: US 2026/0241140 A1
(43) Pub. Date: Aug. 20, 2026

(54) DELIVERY DEVICES FOR BUNDLE BRANCH CARDIAC THERAPY

(71) Applicant: Medtronic, Inc., Minneapolis, MN (US)

(72) Inventors: Zheng WANG, Shanghai (CN); Chunling ZHAO, Shanghai (CN); Juan MENG, Shanghai (CN); Jiayu ZHAO, Shanghai (CN); Jay RASSAT, Otsego, MN (US); Varun BHATIA, Minneapolis, MN (US)

(21) Appl. No.: 19/162,101

(22) PCT Filed: Mar. 9, 2023
(86) PCT No.: PCT/CN2023/080432

§ 371 Date: Sep. 4, 2025

(51) Int. Cl. A61M 25/00; A61M 25/06; A61N 1/372

(52) U.S. Cl. CPC A61M 25/0041; A61M 25/0054; A61M 25/0662; A61N 1/372; A61M 2210/125

(57) ABSTRACT

A catheter (18A) configured to deliver a lead (18) or other secondary device into a patient's heart (12). The catheter (18A) is configured to deliver the secondary device adjacent a portion of the left bundle branch (8a) of the patient via the right ventricle (28). The catheter (18A) includes an elongate tubular member (14). The elongate tubular member (14) includes a proximal end (15a), a distal end (15b) sized for introduction into a patient's body, and a lumen (14A) extending between the proximal end (15a) and the distal end (15b). The elongate tubular member (14) is pre-formed. The elongate tubular member (14) further includes a distal portion (150b) adjacent the distal end (15b) and a proximal portion (99) adjacent the proximal end (15a). The distal portion (150b) defines a curvilinear shape in a relaxed state. The curvilinear shape includes a curved second region (200) lying within a first plane (17) and a curved fourth region (400) lying within a second plane (19).

The present disclosure relates to delivery devices and methods for implantation of implantable medical devices. In particular, the present disclosure relates to delivery devices such as a catheter, for delivery of implantable medical devices, systems, and methods for cardiac therapy, including single chamber or multiple chamber pacing (e.g., dual or triple chamber pacing), atrioventricular synchronous pacing, asynchronous pacing, triggered pacing, cardiac resynchronization pacing, or tachycardia-related therapy.

Implantable medical devices (IMDs), such as cardiac pacemakers or implantable cardioverter defibrillators, deliver therapeutic stimulation to patients' hearts thereby improving the lives of millions of patients living with heart conditions. Conventional pacing techniques involve pacing one or more of the four chambers of a patient's heart 12 as illustrated in FIG. 1, including the left atrium (LA) 33, the right atrium (RA) 26, the left ventricle (LV) 32 and the right ventricle (RV) 28. One common conventional therapeutic pacing technique that treats a slow heart rate, referred to as bradycardia, involves delivering an electrical pulse to a patient's right ventricular tissue. In response to the electrical pulse, both the right and left ventricles contract. However, the heartbeat process may be significantly delayed because the pulse travels from the right ventricle through the left ventricle. The electrical pulse passes through the muscle cells that are referred to as myocytes. Myocyte-to-myocyte conduction may be very slow. Delayed electrical pulses can cause the left ventricle to be unable to maintain synchrony with the right ventricle.

Over time, the left ventricle can become significantly inefficient at pumping blood to the body. In some patients, heart failure can develop such that the heart is too weak to pump blood to the body. Heart failure may be a devastating diagnosis since, for example, fifty percent of heart failure patients have a life expectancy of five years or less. Another possible cause of heart failure is due to atrial fibrillation, which is an irregular and often very rapid heart rhythm or arrhythmia. During atrial fibrillation, the atria of the heart can beat out of sync with the ventricles of the heart because of the arrythmia of the atria, which can lead to blood clots in the heart and increase the risk of stroke or heart failure, for example.

To avoid potential development of heart failure, some physicians have considered alternative pacing methods that involve the cardiac conduction system. Pacing the cardiac conduction system may quickly conduct electrical pulses (for example, akin to a car driving on a highway), whereas pacing cardiac muscle, or myocardial, tissue may more slowly conduct electrical pulses (for example, akin to a car driving on a dirt road).

The cardiac conduction system includes the sinoatrial node 1, atrial internodal tracts 2, 4, 5 (i.e., anterior internodal 2, middle internodal 4, and posterior internodal 5), atrioventricular node 3, His bundle 13A (also known as the atrioventricular bundle or bundle of His), left bundle branch 8a, and right bundle branch 8b as shown in FIG. 1. The arch of aorta 6 and the Bachman's bundle 7 are also shown in FIG. 1. The sinoatrial node 1, located at the junction of the superior vena cava and right atrium, is considered to be the natural pacemaker of the heart as it continuously and repeatedly emits electrical impulses. The electrical impulses spread through the muscles of right atrium 26 to left atrium 33 to cause synchronous contraction of the atria. The electrical impulses are also carried through atrial internodal tracts to the atrioventricular node 3—the sole connection between the atria and the ventricles. The conduction through the atrioventricular node or atrioventricular nodal tissue takes longer than through the atrial tissue, which results in a delay between the atrial contractions and the start of the ventricular contractions. The atrioventricular delay, which is the delay between atrial contractions and ventricular contractions, allows the atria to empty blood into the ventricles. Then, the valves between the atria and ventricles close in conjunction with ventricular contraction via branches of the bundle of His. The bundle of His, or His bundle, 13A is located in the membranous atrioventricular septum near the annulus of the tricuspid valve. The His bundle 13A splits into the left and right bundle branches 8a, 8b and are formed of specialized fibers called “Purkinje fibers” 9. The Purkinje fibers 9 may be described as being capable of rapidly conducting an action potential down the ventricular septum (VS), spreading the depolarization wavefront quickly through the remaining ventricular myocardium, and producing a coordinated contraction of the ventricular muscle mass.

Patients with a conduction system abnormality, such as poor AV node conduction or poor SA node function, may receive an IMD, such as a pacemaker, to restore a more normal heart rhythm and AV synchrony. Some types of IMDs, such as cardiac pacemakers, implantable cardioverter defibrillators (ICDs), or cardiac resynchronization therapy (CRT) devices, provide therapeutic electrical stimulation to a heart of a patient via electrodes on one or more implantable endocardial, epicardial, or coronary venous leads that are positioned in or adjacent to the heart. The therapeutic electrical stimulation may be delivered to the heart in the form of pulses or shocks for pacing, cardioversion, or defibrillation. In some cases, an IMD may sense intrinsic depolarizations of the heart, and control the delivery of therapeutic stimulation to the heart based on the sensing.

Cardiac arrhythmias may be treated by delivering electrical shock therapy for cardioverting or defibrillating the heart in addition to cardiac pacing, for example, from an ICD, which may sense a patient's heart rhythm and classify the rhythm according to an arrhythmia detection scheme to detect episodes of tachycardia or fibrillation.

Arrhythmias detected may include ventricular tachycardia (VT), fast ventricular tachycardia (FVT), ventricular fibrillation (VF), atrial tachycardia (AT) and atrial fibrillation (AT). Anti-tachycardia pacing (ATP), a painless therapy, can be used to treat ventricular tachycardia (VT) to substantially terminate many monomorphic fast rhythms. While ATP is painless, ATP may not deliver effective therapy for all types of VTs. For example, ATP may not be as effective for polymorphic VTs, which has variable morphologies. Polymorphic VTs and ventricular fibrillation (VFs) can be more lethal and may require expeditious treatment by shock.

### SUMMARY

The techniques of this disclosure generally relate to a catheter that guides an implantable medical device or lead for delivery of the device or lead to the correct location in the ventricular septum at or near the left bundle branch (LBB) and at an orientation to deliver pacing at or near the LBB.

It can be difficult to implant a lead close enough to the LBB to effectively pace the LBB, or implanted LBB lead(s) may dislodge over time due to natural movement or due to injury, for example, and left ventricular septal pacing may occur as a result. This is also true for the right bundle branch (RBB) pacing shifting into right ventricular septal pacing. On one hand, for patients whose cardiac conduction systems work normally, septal pacing may be undesirable in some cases. On the other hand, for patients whose cardiac conduction systems do not work normally, septal pacing may be desirable in some cases, such as, for example, when the patient experiences LBB or RBB block that cannot be corrected or bypassed. In other cases for patients whose cardiac conduction systems do not work normally, cardiac conduction system pacing is still desirable, such as, for example, when the LBB or RBB block can be corrected or bypassed. Pacing modes may be chosen based on the individual patient's needs.

Additionally, it can be difficult to implant a lead quickly and efficiently without needing to manipulate the catheter to ensure appropriate lead placement. Steerable and manipulatable catheters thus may be more difficult to use and require a higher level of skill by the medical professional using the catheter to implant a medical device. Additionally, it can be difficult to advance a lead out of a catheter without forces acting on the catheter to push it back from the desired placement, resulting in further difficulty in placing the lead correctly. The stiffness and curvilinear structure of a catheter may help offset such difficulties.

In particular, illustrative devices and methods are described herein related to a catheter or other delivery device configured to deliver a lead or other secondary device into a patient's heart. More specifically, the catheter is designed to deliver the lead at or near the LBB for cardiac conduction system pacing. Such cardiac conduction system pacing may include, for example, LBB pacing or left bundle branch area (LBBA) pacing. The catheter design includes a distal portion that is pre-formed and that is curvilinear in a relaxed state. The distal portion facilitates easier, more efficient LBB placement without the need for manipulation of the catheter to ensure proper placement.

One illustrative catheter may be configured to deliver a lead or other secondary device into a patient's heart and adjacent a portion of the left bundle branch of the patient via the right ventricle. The catheter may include an elongate tubular member including a proximal end, a distal end sized for introduction into a patient's body, and a lumen extending therebetween. The elongate tubular member may be pre-formed. The elongate tubular member may include a distal portion adjacent the distal end and a proximal portion adjacent the proximal end. The distal portion may define a curvilinear shape in a relaxed state. The proximal portion may define a substantially straight or straight shape in a relaxed state.

The distal portion may further include a curved second region defining a second radius and a second angle lying within a first plane. The distal portion may further include a curved fourth region distal to the curved second region and defining a fourth radius and a fourth angle lying within a second plane different than the first plane. The first plane and the second plane may intersect at a plane intersect angle between about 50 degrees to about 110 degrees. The second radius may be about 25 mm to about 115 mm. The fourth radius may be about 10 mm to about 35 mm. The second radius may be greater than the fourth radius.

In some embodiments, the distal portion may further include a first region distal to the proximal portion and defining a first length lying within the first plane. The distal portion may further include a third region distal to the curved second region and defining a third length lying within the first plane. The distal portion may further include a curved sixth region proximal to the first region and defining a sixth radius and a sixth angle lying within the first plane. The distal portion may further include a substantially straight seventh region distal to the curved sixth region and proximal to the first region and defining a seventh length lying within the first plane. The sixth radius may be about 40 mm to about 80 mm. The second radius and the sixth radius may each be greater than the fourth radius.

In some embodiments, the distal portion may further include a fifth region distal to the curved fourth region and proximal to the distal end and defining a fifth length lying within the second plane. The proximal portion may be stiffer than the distal portion. The first region and the curved second region may be stiffer than the third region, the curved fourth region, and the fifth region. Stiffness may be measured in various ways, including, for example, a three points bending test. Standard ISO 180 and ASTM D790 three points bending tests may be used to determine flexural stress and strain, flexural modulus, flexural strength, and flexural stress at break and at 5% strain for rigid and semi-rigid plastics and other materials.

One illustrative method may include advancing a catheter (e.g., such as the catheter described herein) toward a selected location adjacent a portion of the left bundle branch of a patient via the right ventricle. The catheter may include a pre-formed elongate tubular member extending from a proximal end to a distal end and defining a lumen extending therebetween. The elongate tubular member may include a proximal portion adjacent the proximal end and a distal portion adjacent the distal end. The distal portion may define a curvilinear shape in a relaxed state. The proximal portion may define a substantially straight or a straight shape in a relaxed state. The distal portion may further include a curved second region defining a second radius and a second angle lying within a first plane. The distal portion may further include a curved fourth region distal to the curved second region and defining a fourth radius and a fourth angle lying within a second plane different than the first plane. The first plane and the second plane may intersect at a plane intersect angle between about 50 degrees to about 110 degrees. The second radius may be about 25 mm to about 115 mm. The fourth radius may be about 10 mm to about 35 mm. The second radius may be greater than the fourth radius. The method may further include advancing a medical device through the lumen and out the distal end of the elongate body to the selected location for at least one of therapy delivery or sensing. The curvilinear shape and a stiffness of the catheter may promote advancement of the medical lead without relative movement of the catheter during advancement of the medical lead.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a schematic diagram of a heart and conduction system of a patient.

FIG. 2 is a conceptual diagram of an illustrative catheter that is configured to deliver a lead adjacent a portion of the left bundle branch via the right ventricle.

FIG. 3A is a conceptual diagram illustrating an illustrative therapy system that is configured to provide cardiac conduction system pacing therapy to the left bundle branch using a single lead placed in the right ventricle.

FIG. 3B is a close-up view of the lead in the patient's heart of FIG. 3A.

FIG. 4A is a perspective view of multiple identical catheters.

FIG. 4B is another perspective view of a single catheter of FIG. 4A.

FIG. 4C is a partial elevation view of the catheter of FIGS. 4A-B.

FIG. 5 is a partial perspective view of the catheter of FIGS. 4A-C.

FIG. 6A is another partial perspective view of the catheter of FIGS. 4-5.

FIG. 6B is a conceptual diagram of the catheter of FIGS. 4-6A during device implantation into a heart.

FIG. 7A is another partial elevation view of the catheter of FIGS. 4-6.

FIG. 7B is another partial perspective view of the catheter of FIGS. 4-7A.

FIG. 8A is a partial perspective view of another catheter.

FIG. 8B is a conceptual diagram of the catheter of FIG. 8A during device implantation into a heart.

FIG. 9A is another partial elevation view of the catheter of FIG. 8.

FIG. 9B is another partial perspective view of the catheter of FIGS. 8-9A.

FIG. 10 is a block diagram of an illustrative method of implanting a medical lead using one of the catheters of FIGS. 4-9.

### DETAILED DESCRIPTION

In the following detailed description of illustrative embodiments, reference is made to the accompanying figures of the drawing that form a part hereof, and in which are shown, by way of illustration, specific embodiments that may be practiced. It is to be understood that other embodiments may be utilized, and structural changes may be made without departing from (e.g., still falling within) the scope of the disclosure presented hereby.

Delivery devices and methods are described herein which deliver medical devices at or near the LBB from the right ventricle. Single, dual, and/or triple chamber medical devices are available which can be deliverable to the patient using the delivery devices and methods. The medical devices can include a transvenous atrial lead carrying electrodes that may be placed in the right atrium, a transvenous ventricular lead carrying electrodes that may be placed in the right ventricle via the right atrium, a coronary sinus lead that may be placed in the left ventricle via the coronary sinus, and a ventricle-from-atrium (VfA) lead that may be placed in the right atrial septum between the right atria and the left ventricle to pace the left ventricle. Such devices may be implanted using the delivery devices and methods of the present application.

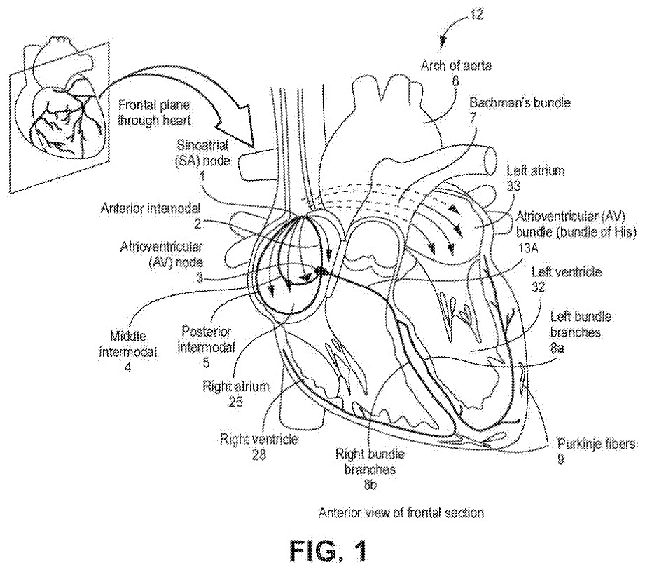

Illustrative systems, devices, and methods shall be described with reference to FIGS. 1-10. It will be apparent to one skilled in the art that elements or processes from one embodiment may be used in combination with elements or processes of the other embodiments, and that the possible embodiments of such systems, devices, and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

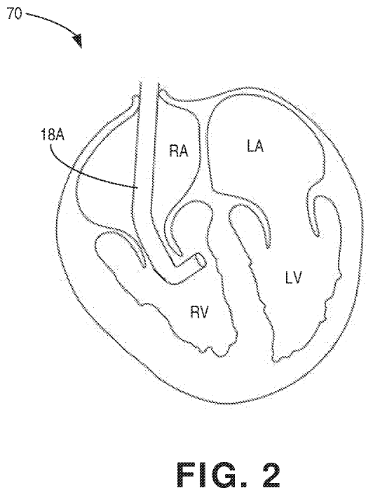

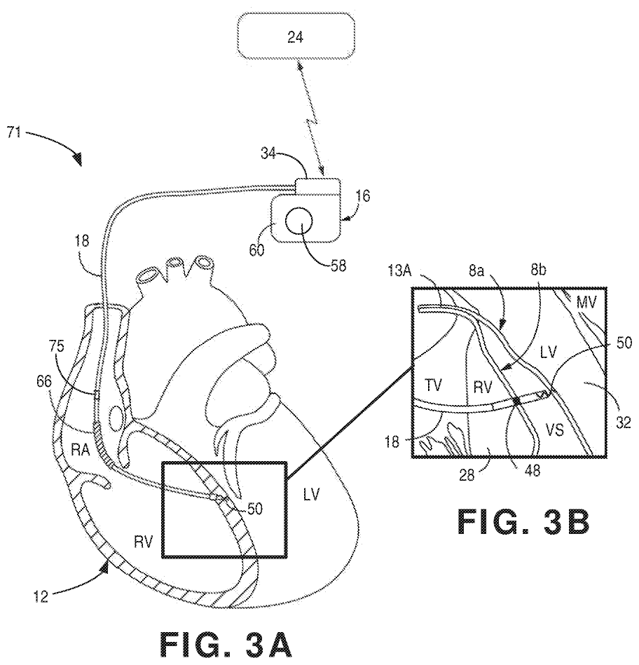

FIG. 1 depicts a schematic diagram of a heart 12 and FIGS. 2-3B depict conceptual diagrams showing illustrative delivery system 70 including a catheter 18A that may be used to deliver a medical device or a therapy system 71 to the heart 12 of a patient. The patient ordinarily, but not necessarily, will be a human. In one or more embodiments, therapy system 70 may include the catheter 18A as discussed herein. The catheter 18A is intended to allow passage of a medical device such as a lead through an inner lumen or passageway of the catheter, so that a user may deliver the device, through the lumen of catheter 18A, to a target site within a patient's body.

The catheter 18A may include various components as discussed further herein, including an in-line hub, which may further include an integrated valve, a flush port, etc. Radiopaque markers may be placed on or coupled to catheter 18A for fluoroscopic of ultrasonic visualization of the catheter 18A once it is introduced into a patient's body. The radiopaque markers may be placed at a distal end of the catheter 18A as discussed herein.

The hub 13 may be constructed of two or more separable parts or may be a single part. An elongate tubular member 14 of the catheter 18A may be couplable to the hub 13 (e.g., via interference fit, using adhesive or welding, etc.). An integrated valve or seal member (not shown) may be designed to seal around the medical device or lead as it passes through the catheter 18A, and may advantageously reduce or prevent leakage of bodily fluids outside the catheter 18A.

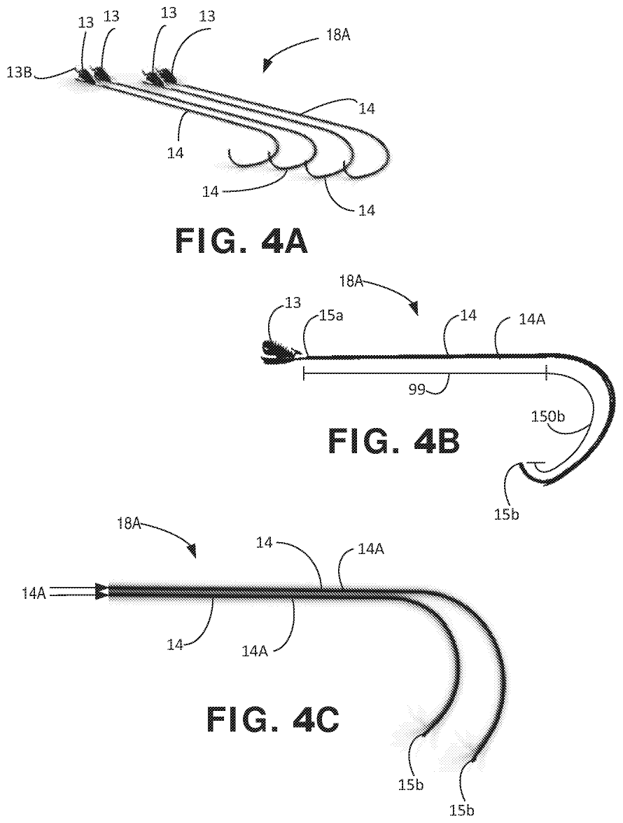

A flush port or other in-line port 13B (as illustrated in FIG. 4A) may include a pierceable or otherwise openable opening into the hub and elongate tubular member 14 such that a syringe or other device may connect to the port (e.g., using a luer lock, needle, etc.). This may advantageously allow introduction or removal of content in the catheter 18A or in the patient's body. Further external engagement members may be coupled to the hub 13, as described in U.S. Pat. App. Pub. No. 2012/0029480 A1, entitled “Catheter Apparatus,” published on Feb. 2, 2012, and incorporated by reference in its entirety.

The hub 13 may include or may itself be as a handle for the user to grasp during operation of the catheter 18A. For example, the hub 13 may be constructed with a tapered profile with a center fin to make it easier to grasp. The hub 13 may also advantageously provide a more comfortable hand position during operation. Further, the hub 13 may include a hub orientation marker (not shown). The hub orientation marker may identify to a user the correct orientation to position or hold the hub in, or to position or hold the device in, such that the alignment direction, the orientation, or the position of the distal end may be correctly and more easily and efficiently reached. The hub orientation marker may take the form of a printed, colored, or visible marker, or may take the form of a tactile, material, or physically discernable marker. The orientation marker may be placed anywhere on, near, or adjacent to the hub 13.

The catheter 18A and the elongate tubular member 14 may be sized to particularly accommodate passage of devices or system that have outer diameters ranging from approximately 0.01 inches to approximately 0.10 inches. In alternative embodiments, the elongate tubular member 14 may be sized to accommodate passage of device or systems that are sized between about 1 French (Fr) and 20 Fr. Thus, the inner diameter of the elongate tubular member 14 may be sized to accommodate the range of devices which may pass through the catheter 18A. In some embodiments, for example, the inner diameter of the catheter body may be between about 3 Fr and 7 Fr. In one embodiment, the inner diameter may be about 5 Fr to about 6 Fr.

One example of a catheter (e.g., designed to deliver a His bundle pacing lead) may include the C315 Delivery Catheter. A description of the C315 Delivery Catheter is found in the Medtronic model C315 Delivery Catheter instructions for use (2016), incorporated herein by reference in its entirety. An example of a deflectable delivery catheter (e.g., designed to deliver a His bundle pacing lead) can be the SELECTSITE™ C304 Delivery Catheter. A description of the SELECTSITE™ C304 Delivery Catheter is found in the Medtronic model SELECTSITE™ C304 Delivery Catheter manual (2018) and instructions for use (2010), both incorporated herein by reference in its entirety. One example of a delivery catheter apparatus may be found in U.S. Pat. App. Pub. No. 2015/0065872 A1, entitled “Delivery Catheter Apparatus and Methods,” published Mar. 5, 2015.

The therapy system 71 may include IMD 16, which is coupled to a cardiac conduction pacing therapy lead 18 (e.g., left bundle branch pacing lead) and a programmer 24. The IMD 16 may be, for example, an implantable pacemaker, cardioverter, and/or defibrillator that provides electrical pulses to the heart 12 via electrodes coupled to the cardiac conduction pacing therapy lead 18. Further non-limiting examples of the IMD 16 include the following: a pacemaker with one or more medical leads, a cardiac resynchronization therapy (CRT) device, an implantable cardioverter-defibrillator (ICD), an intracardiac device, etc. IMD 16 may further include a housing 60 and may include one or more housing electrodes, such as housing electrode 58, which may be formed integrally with an outer surface of a hermetically sealed housing 60 of the IMD 16 or otherwise coupled to the housing 60. The cardiac conduction pacing therapy lead 18 may be electrically coupled to a stimulation generator, a sensing module, or other modules of IMD 16 via a connector block 34.

The cardiac conduction pacing therapy lead 18 may extend into the heart 12 of the patient to sense electrical activity of the heart 12 and/or deliver electrical stimulation to the heart 12. In the example shown in FIG. 3A, the cardiac conduction system pacing therapy lead 18 extends through one or more veins and the vena cava, the right atrium, through the tricuspid valve and into the right ventricle 28 of the heart 12 to pace the cardiac conduction system (e.g., within the ventricular septal wall, proximate and/or in direct contact with the left bundle branch 8a, proximate and/or in direct contact with the right bundle branch 8b, proximate and/or in direct contact with the His bundle 13A, etc.). The cardiac conduction system pacing therapy lead 18 may be positioned for positioning electrodes 48, 50 near, adjacent, on, within, or around the RBB, LBB (respectively) for sensing electrocardiogram signals and pacing the cardiac conduction system. The cardiac conduction system pacing therapy lead 18 is shown with a ring electrode 48 and a helix tip electrode 50 that may be selected in various bipolar pacing electrode pairs for pacing the RBB and the LBB (respectively) and for sensing RBB and LBB electrocardiogram signals (respectively). In alternative embodiments, the cardiac conduction system pacing therapy lead 18 is also used to pace the RA using an electrode 75 (shown in FIG. 3A) or is used to pace the RA in addition to the cardiac conduction system. In further alternative embodiments, the cardiac conduction pacing therapy lead 18 may also include elongated electrode 66 (shown in FIG. 3A), which may take the form of a coil. The IMD 16 may deliver defibrillation shocks to the heart 12 via the elongated electrode 66 and the housing electrode 58.

One example of a cardiac conduction system pacing therapy lead (e.g., a LBB lead) can be the SELECTSECURE™ 3830 (Medtronic, Inc.). A description of the SELECTSECURE™ 3830 is found in the Medtronic model SELECTSECURE™ 3830 manual (2013), incorporated herein by reference in its entirety. The SELECTSECURE™ 3830 includes two conductors without lumens.

As used herein, cardiac conduction system pacing therapy refers to any techniques that are configured to deliver pacing therapy (e.g., pacing pulses, electrical stimulation, etc.) to the cardiac conduction system including, e.g., the His bundle 13A, the left bundle branch 8a, the right bundle branch 8b, etc., to initiate activation.

Illustrative IMDs may be described as delivering one or both of conventional pacing therapy and cardiac conduction system pacing therapy. Conventional, or traditional, pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. For instance, conventional pacing therapy may deliver pacing pulses directly into the muscular heart tissue (e.g., myocardial tissue) that is to be depolarized to provide the contraction of the heart. For example, conventional left ventricular pacing therapy may utilize a left ventricular coronary sinus lead that is implanted so as to extend through one or more veins, the vena cava, the right atrium 26, and into the coronary sinus to a region adjacent to the free wall of the left ventricle 32 of the heart 12 so as to deliver pacing pulses to the myocardial tissue of the free wall of the left ventricle 32.

Illustrative cardiac conduction system pacing therapy may be described in, for example, U.S. Pat. App. Pub. No. 2019/0111270 A1 entitled “His Bundle and Bundle Branch Pacing Adjustment” published on Apr. 18, 2019. Illustrative left ventricular septal pacing may be described in, for example, U.S. patent application Ser. No. 16/521,000 entitled “AV Synchronous Septal Pacing” filed on Jul. 24, 2019.

Cardiac conduction system pacing may include at least one of His bundle pacing, LBB pacing, and RBB pacing. Bundle branch pacing may bypass the pathological region and may have a low and stable pacing threshold. In some embodiments, only one of the left bundle branch or the right bundle branch may be paced using one or more pacing leads. In further embodiments, both bundle branches may be paced at the same time (e.g., dual bundle branch pacing), which may mimic intrinsic activation propagation via the His bundle-Purkinje conduction system, e.g., paced activation propagates via both bundle branches to both ventricles for synchronized contraction. His bundle pacing, on the other hand, typically paces the His bundle proximal to the bundle branches. In some embodiments, the IMD 16 may be coupled to one, two, or more electrodes located in one or more bundle branches configured for bundle branch pacing. In some embodiments, the IMD 16 may be an intracardiac pacemaker configured to pace one or more portions of the cardiac conduction system such as one or both of the bundle branches.

FIGS. 3A-3B show the patient's heart 12 implanted with cardiac conduction system pacing lead 18 to deliver bundle branch pacing according to one example of the therapy system 71. The cardiac conduction system therapy lead 18 is positioned, or located, through the tricuspid valve into the RV 28 and implanted in the interventricular septum, e.g., about 1 to 2 centimeters in an apical direction away from the RA (as illustrated in FIGS. 3A-B). FIG. 3B is a close-up view of the cardiac conduction system therapy lead 18 in the patient's heart 12 of FIG. 3A. In some embodiments, the cardiac conduction system therapy lead 18 may be the only lead implanted in the heart 12. In other embodiments, there may be leads in addition to the cardiac conduction system therapy lead 18 implanted in the heart 12, such as one or more leads implanted in the right atrium, right ventricle apex, and left ventricle via the coronary sinus.

The configuration of therapy system 71 illustrated in FIG. 3A-B is merely an example. In other examples, a therapy system may include epicardial leads and/or patch electrodes instead of or in addition to the cardiac conduction system therapy lead 18 or other configurations shown or described herein or incorporated by reference. Further, the IMD 16 need not be implanted within patient. As such, it is to be understood that the illustrative therapy systems described herein may include any suitable number of leads coupled to IMD 16, and each of the leads may extend to any location within or proximate to the heart 12. For example, illustrative therapy systems may include a single transvenous lead located as illustrated in FIGS. 3A-3B, or two or more transvenous leads located in various chambers.

The illustrative devices and methods described herein may provide an efficient way to implant a lead into a patient proximate a portion of the LBB using a pre-formed, curvilinear, multiplanar catheter as illustrated in FIGS. 4-9.

In at least one embodiment, and as illustrated in FIGS. 4A-C and 6A, the catheter 18A may be configured to deliver a lead or other secondary device, such as cardiac conduction system pacing lead 18, into a patient's heart and adjacent a portion of the left bundle branch of the patient via the right ventricle. The catheter 18A may include an elongate tubular member (also known as a catheter body) 14 including a proximal end 15a and a distal end 15b. The elongate tubular member 14 may be sized for introduction into a patient's body. The catheter 18A may further include a lumen 14A extending between the proximal end 15a and the distal end 15b. The lumen 14A may be configured to house a medical device such as cardiac conduction system pacing lead 18 (FIGS. 3A-B). The medical device will be bounded by the structure of the lumen 14A of the catheter 18A during implantation of the medical device into the patient.

The elongate tubular member 14 may be pre-formed. For example, the elongate tubular member 14 may be constructed to hold a certain form or shape when in a relaxed state or when in a flexed state. In one or more embodiments, the catheter 18A may be preformed to retain a shape when in a relaxed state. A preformed catheter may thus be designed to require less operator manipulation compared to steerable, non-preformed catheters, because the preformed shape will place the distal end 15b of the catheter 18A nearer to the desired location without requiring steering or manual placement of the distal end 15b.

The catheter 18A may further include a distal portion 150b (illustrated in FIGS. 4B, 6A, and 8A) adjacent the distal end 15b. Distal portion 150b may include any and all regions 100-700 as described further herein. The catheter 18A may further include a proximal portion 99 adjacent the proximal end 15a. The distal portion 150b may define a curvilinear and multiplanar shape in a relaxed state, as discussed herein. The proximal portion 99 may define a substantially straight or straight shape in a relaxed state. Additionally, the catheter 18A may include the hub 13 at the proximal end 15a. The hub 13 may include an integrated valve, a flush port, etc., similar to Medtronic Delivery Catheter C315.

The catheter 18A may be sized such that, in its preformed shape, the distal end 15b will be positioned substantially perpendicular to the ventricular septum in the RV and adjacent to the LBB for successful implantation of an LBB lead (e.g., cardiac conduction system pacing lead 18). This may be true for a variety of patients with a variety of anatomies (e.g., pacemaker indicated patients, ICD indicated patients, CRT indicated patients, Heart Failure Class III and Class IV patient anatomies, etc.). A study of approximately 80 CT scans and 22 different model heart anatomies resulted in an understanding that most of the heart configurations may be serviced by two differently sized catheters 18A, depending at least in part on the point of access and the curvatures described herein. In alternative embodiments, more or fewer than two sizes of catheter 18A may be used based on the ranges for the various regions as further described herein. The catheter 18A may include a range of radius and curve angle values for different sections, segments, or regions of the catheter 18A. The range of radius and curve angles may differ between the larger and smaller catheter embodiments.

For heart anatomies with a larger sized RA, for example, in a sample set of the patient population, the RA volume was between about 55 milliliters (ml) and about 316 ml. The average volume was about 165 ml. The RA short axis diameter was between about 2.5 centimeters (cm) and about 7.5 cm. The RA long axis diameter was between about 5 cm and about 10 cm. For patients with a larger-sized RA, a larger catheter 18A as described herein may be preferred. In alternative embodiments, a smaller catheter 18A as described herein may still be preferred for patients with a larger-sized RA.

For heart anatomies with a relatively normal sized RA, for example, in a sample set of the patient population, the RA volume was between about 40 ml and about 140 ml. The average volume was about 97 ml. The RA short axis diameter was between about 3.5 cm and about 4.5 cm. The RA long axis diameter was between about 5 cm and about 7.5 cm. For patients with a more typically sized RA, a smaller catheter 18A as described herein may be preferred. In alternative embodiments, a larger catheter 18A as described herein may still be preferred for patients with a more typically sized RA.

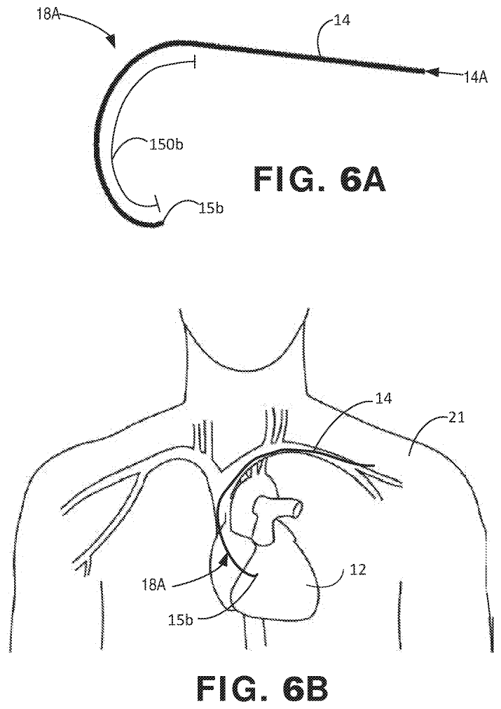

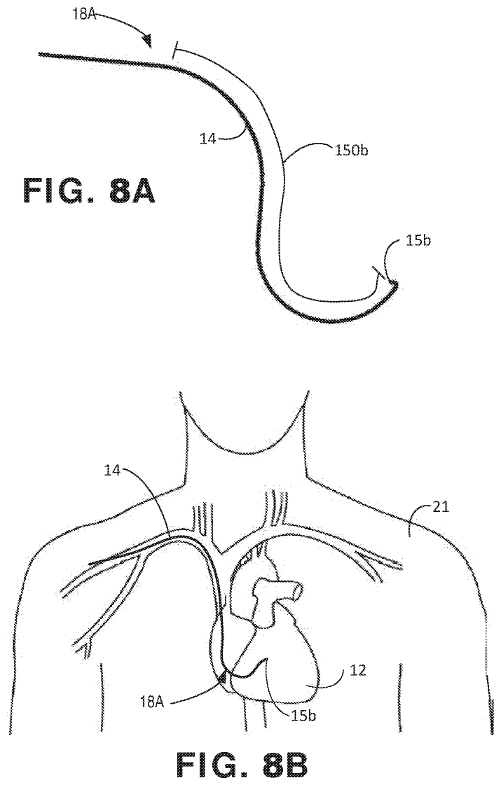

The catheter 18A may be configured for introduction from the right or left side of a patient 21. In one or more embodiments, the catheter 18A may be configured to be implanted from at least one of the left cephalic vein, the axillary vein, and the left subclavian vein of the patient through the right atrium and into the right ventricle of the patient's heart, as illustrated in FIG. 6B. Such access points may be collectively referred to as the left access point herein. Although certain embodiments are shown with respect to FIGS. 6B and 8B, discussed below, any embodiment of catheter 18A may be implanted through the vasculature as illustrated (e.g., with or without the additional curves, as discussed further herein). In one embodiment, the catheter 18A may include a range of radius and curve angle values for different sections, segments, or regions of the catheter 18A for the smaller heart anatomy. In another embodiment, the catheter 18A may include a range of radius and curve angle values for different sections, segments, or regions of the catheter 18A for the larger heart anatomy.

In one or more embodiments, the catheter 18A may be configured to be implanted from at least one of the right cephalic vein, the right subclavian vein, and the axillary vein of the patient through the right atrium and into the right ventricle of the patient's heart 12, as illustrated in FIG. 8B. Such access points may be collectively referred to as the right access point herein. Although certain embodiments are shown with respect to FIGS. 6B and 8B, any embodiment of catheter 18A may be implanted through the vasculature as illustrated (e.g., with or without the additional curves, as discussed further herein). In one embodiment, the catheter 18A may include a range of radius and curve angle values for different sections, segments, or regions of the catheter 18A for the smaller heart anatomy. In another embodiment, the catheter 18A may include a range of radius and curve angle values for different sections, segments, or regions of the catheter 18A for the larger heart anatomy.

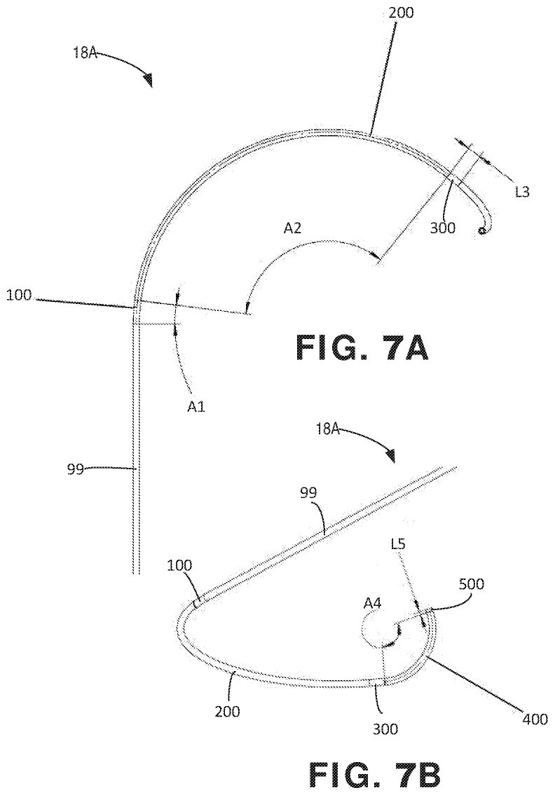

As illustrated in FIGS. 7A and/or 7B, the distal portion may include one or more of a first region 100, a curved second region 200, a third region 300, a curved fourth region 400, and an optional fifth region 500. These regions 100-500 may advantageously provide more efficient and more accurate positioning of the distal end 15b of the catheter 18A adjacent a portion of the LBB, at least because the user may not need to manipulate or steer the catheter as much or at all. These regions 100-500 may advantageously be used in the catheter 18A when the catheter 18A is advanced from either the left or the right sides of the patient. The radii and angles discussed herein for each region 100-400 may be identical for both left side access and right side access of catheter 18A.

For example, facing the patient, and introducing the catheter 18A from the left side access, if the curved second region 200 curves counter-clockwise relative to the center of an imaginary circle, then from the right side access, the curved second region 200 also curves counter-clockwise relative to the same center of the same imaginary circle. Further, for example, the regions 100-400 may be positioned such that they are concaved towards the heart and may be positioned such that they generally curve about the right ventricle of the heart. The magnitude of the length of each region 100-500 may also be identical for both the left and right side access. For both left and right side access, the distal end 15b of the catheter 18A is positioned adjacent the LBB. This is a result of, for example, the difference in left and right side initial access points and any additional regions besides regions 100-400.

Inclusion or of the first region 100 may advantageously provide a better anatomical fit for more efficient and accurate placement of the distal end 15b adjacent the LBB for a majority of heart sizes. Inclusion of the first region 100 may provide a stiffness or hardness which may advantageously provide optimal rigidity and structure during implantation. The inclusion of the first region 100 of catheter 18A may allow the catheter 18A to be moved smoothly along the vessel when the catheter 18A is advanced to the right atrium. Additionally, the gradual transition from the substantially straight proximal section 99 to the curved second region 200, and the combination of the first region 100 and the curved second region 200, allows for optimized geometry for ease or efficiency of implantation, allows for ease of manufacturing, and also allows for a separate first region 100 which may have a different stiffness (or other variable, such as length or material) compared to the immediately the surrounding regions on the proximal and distal ends of the first region 100. The inclusion of the first region 100, the gradual transition, and the stiffness may be optimized for implantation of the catheter 18A in a patient.

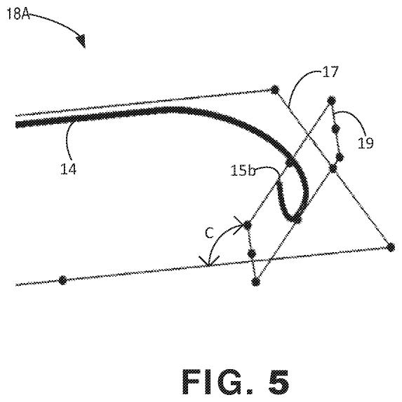

The first region 100 may define a first radius R1 (not shown) and a first angle A1 (shown in FIG. 7A) lying within a first plane 17 (shown in FIG. 5). R1 may be defined as the radius of an imaginary circle extending through the first region 100. A1 may be defined as the angle within that same imaginary circle which encompasses the first region 100. The first region 100 may have a first length L1 (not shown) based on the R1 and A1. The first plane 17 may be positioned substantially or generally parallel to the coronal plane, which bisects the human body into a ventral (front) section and a dorsal (back) section. The first plane 17 may be positioned such that it is substantially parallel to the coronal plane and may bisect the left side access point (e.g., for embodiments which are introduced from the left side), and/or may bisect the right side access point (e.g., for embodiments which are introduced from the right side). The first region 100 may curve along the first plane 17 in a direction that is concave towards the heart. For example, the first region 100 may curve in a direction around the heart. The first region 100 may have compound or constant curvature such that the R1 may be a compound radius or constant radius (e.g., constant or nearly constant) falling within the range of the radii described. In one embodiment, the first region 100 has a substantially constant curvature.

The first radius R1 may be between about 50 millimeters (mm) and about 150 mm. A larger sized catheter 18A introduced from either the left or right side of the patient may include an R1 between about 80 mm and about 120 mm. A smaller sized catheter 18A introduced from either the left or right side of the patient may include an R1 between about 80 mm and about 120 mm. In at least one embodiment, R1 may be about 100 mm. In other embodiments for either size and either introduction side, R1 may be between about 75 mm and about 130 mm, between about 95 mm and about 110 mm, etc.

In some embodiments, the first region 100 is curved and the first angle A1 may be between about 1 degree and about 60 degrees for a catheter 18A introduced from either the left or right side of the patient. In some embodiments, the first region 100 may be considered straight (or substantially straight) within manufacturing tolerances without an intended curvature or radius. The first region 100 allows for variability in the curvature of the catheter 18A. Catheter 18A may be optimized based on patient needs and desirability. In at least one embodiment, A1 may be about 30 degrees. In other embodiments, A1 may be between about 5 degrees and 60 degrees, between about 10 degrees and about 50 degrees, between about 20 degrees and about 40 degrees, etc.

In one or more embodiments, the distal portion 150b further includes a curved second region 200. The curved second region 200 may be distal to the first region 100 such that the curved second region 200 is closer to the distal end 15b than the first region 100 and/or the proximal portion 99. The curved second region 200 may be operatively coupled to the first region 100 in embodiments with the first region 100. For example, the curved second region 200 may be positioned adjacent to first region 100 within manufacturing tolerances. The curved second region 200 may be operatively coupled to the proximal portion 99 in embodiments without the first region 100, and may be positioned adjacent to the proximal portion 99 within manufacturing tolerances. The curved second region 200 may define a second radius R2 (not shown) and a second angle A2 lying within the first plane 17. R2 may be defined as the radius of an imaginary circle extending through the curved second region 200. A2 may be defined as the angle within that same imaginary circle which encompasses the curved second region 200. The curved second region 200 may have compound or constant curvature such that R2 may be a compound radius or substantially constant (e.g., constantly or nearly constant) radius falling within the range of radii described. In one embodiment, the curved second region 200 has a substantially constant curvature.

Inclusion or of the curved second region 200 may advantageously provide a better anatomical fit for more efficient and accurate placement of the distal end 15b adjacent the LBB for a majority of heart sizes. The curved second region 200 may be sized to ensure that the catheter 18A can reach the target location, and further may be sized to ensure that the catheter 18A does reach the target location. The unique R2 and A2 chosen and used may be optimized for a specific patient or type of patient. The curved second region 200 of catheter 18A may allow the catheter 18A to be moved smoothly along the vessel when the catheter 18A is advanced to the right atrium. Additionally, the gradual transition from the substantially straight proximal section 99 and/or the first region 100 to the curved fourth region 400 and/or the third region 300 allows for optimized geometry for ease or efficiency of implantation, allows for ease of manufacturing, and also allows for a separate curved second region 200 which may have a different stiffness (or other variable, such as length or material) compared to the immediately the surrounding regions on the proximal and distal ends of the curved second region 200. The inclusion of the curved second region 200, the gradual transition, and the stiffness may be optimized for implantation of the catheter 18A in a patient.

R1, A1, R2, and A2 may be oriented such that the first region 100 and the curved second region 200 curve in the same direction in space along first plane 17 (e.g., A1 and A2 are both positive angles within a common x-y plane). This is illustrated in FIGS. 7A-B, where the first region 100 and the curved second region 200 have the same direction of curvature. Thus, the curved second region 200 may curve along the first plane 17 in a direction that is concave towards the heart. For example, the curved second region 200 may curve from the left side of the patient towards the right side of the patient, and further may curve from the head of the patient towards the foot of the patient.

The curved second region 200 may have a second length L2 (not shown) based on the R2 and A2. In some examples, L2 may be greater than L1. The second radius R2 may be between about 20 millimeters (mm) and about 120 mm. A larger sized catheter 18A for larger heart volumes introduced from either the left or right side of the patient may include an R2 between about 40 mm and about 111 mm. In at least one embodiment for the larger sized catheter 18A, R2 may be about 68.5 mm. In other embodiments for the larger sized catheter 18A, R2 may be between about 40 mm and about 111 mm, between about 50 mm and about 90 mm, etc.

A smaller sized catheter 18A introduced from either the left or right side of the patient may include an R2 between about 25 mm and about 85 mm. In at least one other embodiment for the smaller sized catheter 18A, R2 may be about 52 mm. In other embodiments for the smaller sized catheter 18A, R2 may be between about 25 mm and about 85 mm, between about 35 mm and about 70 mm, etc.

The second angle A2 may be between about 80 degrees and about 230 degrees for a catheter 18A introduced from either the left or right side of the patient. A larger sized catheter 18A introduced from either the left or right side of the patient may include an A2 between about 90 degrees and about 180 degrees. In at least one other embodiment for the larger sized catheter 18A, A2 may be about 129 degrees. In other embodiments for the larger sized catheter 18A, A2 may be between about 95 degrees and about 167 degrees, between about 100 degrees and about 158 degrees, etc.

A smaller sized catheter 18A introduced from either the left or right side of the patient may include an A2 between about 95 degrees and about 225 degrees. In at least one embodiment for the smaller sized catheter 18A, A2 may be about 136 degrees. In other embodiments for the smaller sized catheter 18A, A2 may be between about 105 degrees and about 205 degrees, between about 110 degrees and about 200 degrees, etc.

In one or more embodiments, the distal portion 150b further includes a third region 300. Inclusion of the third region 300 may advantageously provide a better anatomical fit for more efficient and accurate placement of the distal end 15b adjacent the LBB for a majority of heart sizes. Inclusion of the third region 300 may provide a stiffness or hardness which may advantageously provide optimal rigidity and structure during implantation. The inclusion of the third region 300 of catheter 18A may allow the catheter 18A to be moved smoothly along the vessel when the catheter 18A is advanced to the right atrium. Additionally, the gradual transition from the curved second region 200 to the curved fourth region 400 allows for optimized geometry for ease or efficiency of implantation, allows for ease of manufacturing, and also allows for a separate third region 300 which may have a different stiffness (or other variable, such as length or material) compared to the immediately the surrounding regions on the proximal and distal ends of the third region 300. The inclusion of the third region 300, the gradual transition, and the stiffness may be optimized for implantation of the catheter 18A in a patient.

In embodiments with the third region 300, the third region 300 may be distal to the curved second region 200 so that it is closer to the distal end 15b than the curved second region 200. The third region 300 may be substantially straight, straight, or curved. The third region 300 may define a third length L3 lying within the first plane 17. L3 may be greater than L2. The third region 300 may be operatively coupled to the curved second region 200 such that it extends distally from the curved second region 200 along first plane 17. In some embodiments, the third region 300 may be considered straight (or substantially straight) within manufacturing tolerances without an intended curvature or radius.

In alternative embodiments where the third region 300 is curved, the third region 300 may define a third angle A3 and a third radius R3 lying within the first plane 17. R3 may be defined as the radius of an imaginary circle extending through the third region 300. A3 may be defined as the angle within that same imaginary circle which encompasses the third region 300. The third angle A3 may be between about 1 degree and about 10 degrees. In such alternative embodiments, the third region 300 may have compound or constant curvature such that the radius R3 may be a compound radius or constant radius (e.g., constant or nearly constant). In one alternative embodiment, the third region 300 has a substantially constant curvature.

The third length L3 may be between about 1 mm and about 10 mm for a catheter 18A introduced from either the left or right side of the patient. A larger sized catheter 18A introduced from either the left or right side of the patient may include an L3 between about 2 mm and about 8 mm. A smaller sized catheter 18A introduced from either the left or right side of the patient may include an L3 between about 2 mm and about 8 mm. In at least one embodiment, L3 may be about 5 mm. In other embodiments, L3 may be between about 3 mm and about 9 mm, between about 4 mm and about 7 mm, between about 5 mm and about 6 mm, etc.

The curved fourth region 400 (as illustrated in FIGS. 7B and 8B) may be distal to the third region 300 and/or the curved second region 200 so that it is closer to the distal end 15b than the third region 300 and/or the curved second region 200. The curved fourth region 400 may define a fourth radius R4 (not shown) and a fourth angle A4 lying within a second plane 19 different than the first plane 17. The curved fourth region 400 may curve along the second plane 19 in a direction that is generally from the ventral (front) section towards the dorsal (back) section.

A plane intersect angle C (illustrated in FIG. 5) between the first plane 17 and the second plane 19 may be about 50 degrees to about 110 degrees for a smaller or a larger sized catheter 18A introduced from either the left or right side of the patient. In at least one embodiment, C may be about 73.5 degrees. In other embodiments, C may be between about 50 degrees and about 110 degrees, between about 60 degrees and about 100 degrees, between about 70 degrees and about 80 degrees, etc. The first plane 17 may be positioned substantially or generally parallel to the coronal plane, which bisects the human body into a ventral (front) section and a dorsal (back) section. The first plane 17 may be positioned such that it is substantially parallel to the coronal plane and bisects the left access point. The intersect angle C may extend from the first plane 17 towards the dorsal (back) section of the body. The plane intersect angle C is such that, when moving distally along the body of the catheter 18A, the catheter 18A and the curved fourth region 400 project in a direction in space generally towards the back portion and towards an upper portion of the body.

The intersect angle C may advantageously provide a better anatomical fit for more efficient and accurate placement of the distal end 15b adjacent the LBB for a majority of heart sizes for either left or right side access. The intersect angle C may allow the catheter 18A to be moved smoothly along the vessel when the catheter 18A is advanced into and through the right atrium and into the right ventricle. Additionally, the gradual transition from the first plane 17 to the second plane 19 allows for optimized geometry for ease or efficiency of implantation. The inclusion of the intersect angle C the gradual transition may be optimized for implantation of the catheter 18A in a patient such that the distal end 15b is positioned adjacent the LBB.

R4, A4, R2, and A2 may be oriented such that the curved fourth region 400 and the curved second region 200 curve in the same direction if the plane interest angle C were hypothetically 0 degrees, and both regions were curving in space along first plane 17 (e.g., A4 and A2 are both positive angles within a common x-y plane). This is illustrated in FIGS. 7A-B, where the curved fourth region 400 and the curved second region 200 have the same direction of curvature if they are both in the first plane 17.

R4 may be defined as the radius of an imaginary circle extending through the curved fourth region 400. A4 may be defined as the angle within that same imaginary circle which encompasses the curved fourth region 400. The curved fourth region 400 may have a fourth length LA (not shown) based on the R4 and A4. R4 may be less than R2 in some embodiments. L4 may be greater than L1, L3, and/or L5 (discussed further herein). L4 may be less than L1. LA may be less than L2. The curved fourth region 400 may have compound or constant curvature such that R4 may be a compound radius or constant radius (e.g., constant or nearly constant) falling within the range of radii described. In one embodiment, the curved fourth region 400 has a substantially constant curvature.

The fourth radius R4 may be between about 5 mm and about 50 mm for a catheter 18A introduced from either the left or right side of the patient. A larger sized catheter 18A introduced from either the left or right side of the patient may include an R4 between about 10 mm and about 31 mm. A smaller sized catheter 18A introduced from either the left or right side of the patient may include an R4 between about 10 mm and about 31 mm. In at least one embodiment, R4 may be about 17.5 mm. In other embodiments introduced from either the left or right side of the patient, R4 may be between about 11 mm and about 29 mm, between about 12 mm and about 28 mm, etc.

The fourth angle A4 may be between about 45 degrees and about 200 degrees for a catheter 18A introduced from either the left or right side of the patient. A larger sized catheter 18A introduced from either the left or right side of the patient may include an A4 between about 55 degrees and about 175 degrees. A smaller sized catheter 18A introduced from either the left or right side of the patient may include an A4 between about 55 degrees and about 175 degrees. In at least one embodiment, A4 may be about 105 degrees. In other embodiments for either sized catheter 18A introduced from either the left or right side, A4 may be between about 70 degrees and about 140 degrees, between about 95 degrees and about 115 degrees, between about 66 degrees and about 155 degrees, etc.

The first region 100, the curved second region 200, and the third region 300 may all lay substantially within the first plane 17, and the curved fourth region 400 may lay within the second plane 19 for a catheter 18A introduced from either the left or right side of the patient. The first plane 17 and the second plane 19 may position the first region 100, the curved second region 200, the third region 300, and the curved fourth region 400 into the RV of the patient's heart, and the distal end 15b may be positioned in contact with the RV septum at an angle that is substantially perpendicular to the septal wall (e.g., perpendicular, or nearly perpendicular). The planes 17, 19 may also position the distal end 15b adjacent the left bundle branch within the septal wall to help guide a conduction system pacing lead to the target treatment site. This more easily and more efficiently allows a user to advance an LBB pacing lead for implantation adjacent a portion of the LBB, at least because the user may not need to manipulate or steer the catheter as much or at all.

The proximal portion 99 may be stiffer than the distal portion 150b for a smaller or a larger sized catheter 18A introduced from either the left or right side of the patient. Such stiffness of the device is at least a factor of the material used in the device. The first region 100 and the curved second region 200 may each be stiffer than the third region 300 and the curved fourth region 400. Various possible materials and stiffnesses values are discussed further herein.

Stiffness transitions, or differences in stiffness between the various regions, such as those described herein, may minimize kinking of the elongate tubular member 14 of the catheter 18A. Additionally, stiffness transitions may provide consistent torque, push efficiency (ratio of force applied and resulting motion or advancement of the catheter 18A), and other handling variables. The various stiffnesses of the regions of the catheter 18A may additionally and advantageously provide a better device for physicians to use or manipulate more easily or efficiently. The stiffer segments may advantageously offer more rigidity and support, which may advantageously prevent movement of the catheter 18A in a direction opposite the forces that the catheter 18A places on cardiac tissue. For example, the distal end 15b may contact the ventricular septum, and as a lead or other device is advanced through the catheter 18A and contacts the same cardiac tissue, the catheter 18A may be pushed away from the cardiac tissue due to the forces acting on the catheter 18A, the lead or other device, and the cardiac tissue. The stiffness of the regions may reduce or prevent such pushback. The less stiff regions may advantageously protect the device and make sure the device is safe to contact tissue or vessels.

In one or more embodiments, the distal portion 150b further includes an optional fifth region 500 for a smaller or a larger sized catheter 18A introduced from either the left or right side of the patient. The fifth region 500 may advantageously be used or may be omitted from embodiments to provide an optimized shape, stiffness, or composition to deliver the distal end 15b proximate the LBB. The fifth region 500 may advantageously be straight, substantially straight, or curved in any embodiment to provide an optimized shape, stiffness, or composition to deliver the distal end 15b proximate the LBB.

In embodiments including the fifth region 500, the fifth region 500 may be distal to the curved fourth region 400 so that it is closer to the distal end 15b than the curved fourth region 400. The fifth region 500 may be proximal to the distal end 15b. The fifth region 500 may define a fifth length L5 lying within the second plane 19. The fifth region 500 may be operatively coupled to the curved fourth region 400 such that it extends distally from the curved fourth region 400 along the second plane 19. The fifth region 500 may, in some embodiments, be curved and may have a fifth angle A5 (not shown) that may be about 1 degree to about 10 degrees, and a fifth radius R5 (not shown). A5 may be defined as the angle within that same imaginary circle which encompasses the fifth region 500. In embodiments where the fifth region is any of straight, curved, or substantially straight, L5 may be less than each of L1-L4. L5 may be greater than L3.

In embodiments including the fifth region 500, the fifth length L5 may be between about 0.1 mm and about 10 mm for a catheter 18A introduced from either the left or right side of the patient. A larger sized catheter 18A introduced from either the left or right side of the patient may include an L5 between about 0.5 mm and about 5 mm. A smaller sized catheter 18A introduced from either the left or right side of the patient may include an L5 between about 0.1 mm and about 5 mm. In at least one embodiment, L5 may be about 2 mm. In other embodiments, L5 may be between about 1 mm and about 4 mm, between about 2 mm and about 3 mm, etc.

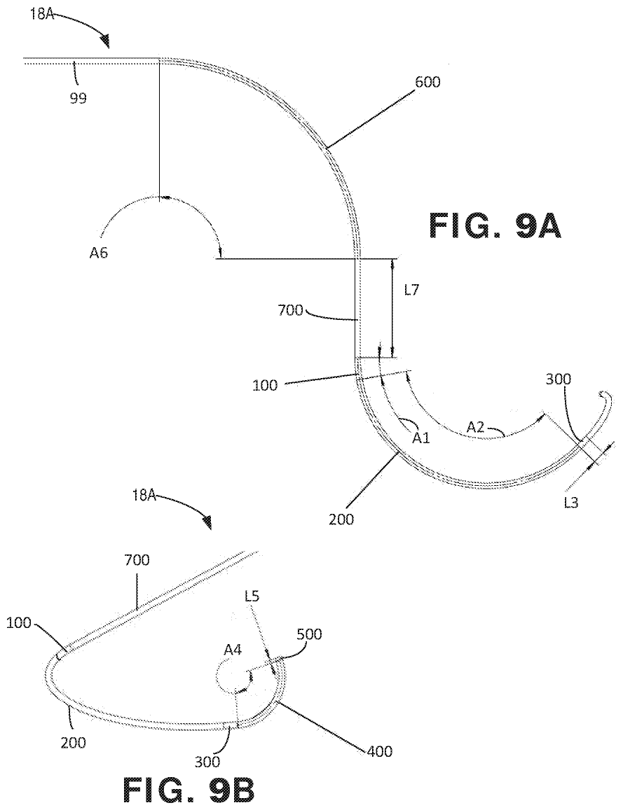

As discussed herein, the catheter 18A for introduction from the left or right side of the patient may include at least the curved second region 200 and the curved fourth region 400, and may alternatively further include the first region 100, the third region 300, and/or the fifth region 500. Additionally, as discussed further herein, in further embodiments, the catheter 18A for introduction from the right side of the patient may also include one or more of a curved sixth region 600 and a substantially straight region 700 as shown in FIGS. 8A and 9A-B. The catheter 18A for introduction from the right side of the patient may include the previously described regions 100-500 as discussed herein and may additionally include the curved sixth region 600 and the substantially straight region 700.

The curved sixth region 600 and the substantially straight region 700 may advantageously allow for easier or more efficient introduction of the catheter 18A from the right side of the patient or may advantageously provide a more accurate placement of the distal end 15b proximate the LBB for the catheter 18A introduced from the right side of the patient. Additionally, the curved sixth region 600 and the substantially straight region 700 may advantageously provide optimal stiffness, component parts and materials, or other mechanical properties which advantageously provides optimal rigidity during introduction of the catheter 18A and which advantageously provides a more accurate placement of the distal end 15b proximate the LBB.

Inclusion or of the curved sixth region 600 and the substantially straight region 700 may advantageously provide a better anatomical fit for more efficient and accurate placement of the distal end 15b adjacent the LBB for a majority of heart sizes. The inclusion of the curved sixth region 600 and the substantially straight region 700 of catheter 18A may allow the catheter 18A to be implanted through the tortuous pathways of the patient's blood vessels to reach the target location. For example, the curved sixth region 600 and the substantially straight region 700 of catheter 18A may allow the catheter 18A to be implanted through the right cephalic vein, or the right subclavian vein, or the axillary vein of the patient, and further may allow the catheter 18A to be implanted through the right atrium and into the right ventricle of the patient's heart. Such implantation may be made easier and more efficient for a user. Inclusion of the curved sixth region 600 and the substantially straight region 700 may provide a stiffness or hardness which may advantageously provide optimal rigidity and structure during implantation. The inclusion of the curved sixth region 600 and the substantially straight region 700 of catheter 18A may allow the catheter 18A to be moved smoothly along the vessel when the catheter 18A is advanced to the right atrium. Additionally, the gradual transition from the substantially straight proximal section 99 to the curved sixth region 600 and the substantially straight region 700 allows for optimized geometry for ease or efficiency of implantation, ease of manufacturing, and also allows for separate curved sixth region 600 and substantially straight region 700 which may have a different stiffness (or other variable, such as length or material) compared to the immediately the surrounding regions on the proximal and distal ends of the curved sixth region 600 and the substantially straight region 700. The inclusion of the curved sixth region 600 and the substantially straight region 700, the gradual transition, and the stiffness may be optimized for implantation of the catheter 18A in a patient. Omission of curved sixth region 600 and the substantially straight region 700 from the catheter 18A may be more advantageous for a left side access point, as discussed herein.

In one or more embodiments of a catheter 18A introduced from the right side of the patient, and as illustrated in FIGS. 8A and 9A-B, the distal portion 150b further includes a curved sixth region 600 can be distal to the proximal portion 99 such that it is closer to the distal end 15b than the proximal portion 99. In one or more embodiments, the curved sixth region 600 is operatively coupled to the proximal portion 99 and extends from the proximal portion 99 towards the distal end 15b. The curved sixth region 600 may define a sixth radius R6 (not shown) and a sixth angle A6. R6 may be defined as the radius of an imaginary circle extending through the curved sixth region 600. A6 may be defined as the angle within that same imaginary circle which encompasses the curved sixth region 600. The curved sixth region 600 may lie within the first plane 17 proximal in relation to, or proximal to, the first region 100. The curved sixth region 600 may have a sixth length L6 (not shown) based on the R6 and A6. R6 may be greater than R4. L6 may be greater than L2. In alternative embodiments, L6 may be less than L2 and may be greater than L7 (discussed further herein). For example, the curved sixth region 600 may curve from the right side of the patient towards the left side of the patient, and further may curve from the head of the patient towards the foot of the patient. The curved sixth region 600 may have compound or constant curvature such that the R6 may be a compound radius or constant radius falling within the range of radii described. In one embodiment, the curved sixth region 600 has a substantially constant curvature.

Additionally, R6, A6, R1, and A1 may be oriented such that the curved sixth region 600 and the first region 100 curve in different directions in space along first plane 17 (e.g., one of A6 and A4 is a positive angle within a common x-y plane, and the other one of A6 and A4 is a negative or a differently valued positive angle within the common x-y plane). This is illustrated in FIGS. 9A-B, where the curved sixth region 600 and the first region 100 have different directions of curvature if they are both in the first plane 17. In alternative embodiments, the curved sixth region 600 and the first region 100 have the same direction of curvature.

Additionally, R6, A6, R2, and A2 may be oriented such that the curved sixth region 600 and the curved second region 200 curve in different directions in space along first plane 17 (e.g., one of A6 and A2 is a positive angle within a common x-y plane, and the other one of A6 and A2 is a negative angle or a differently valued positive angle within the common x-y plane). This is illustrated in FIGS. 9A-B, where the curved sixth region 600 and the curved second region 200 have different directions of curvature if they are both in the first plane 17 such that the combination of curved regions create an “S,” or serpentine, shape.

The sixth radius R6 may be between about 20 mm and about 100 mm. A larger sized catheter 18A introduced from the right side of the patient may include an R6 between about 40 mm and about 80 mm. A smaller sized catheter 18A introduced from the right side of the patient may include an R6 between about 40 mm and about 80 mm. In at least one embodiment, R6 may be about 70 mm. In other embodiments, R6 may be between about 50 mm and about 75 mm, between about 59 mm and about 68 mm, between about 60 mm and 65 mm, etc.

The sixth angle A6 may be between about 70 degrees and about 130 degrees. A larger sized catheter 18A introduced from the right side of the patient may include an A6 between about 80 degrees and about 120 degrees. A smaller sized catheter 18A introduced from the right side of the patient may include an A6 between about 80 degrees and about 120 degrees. In at least one embodiment, A6 may be about 100 degrees. In other embodiments, A6 may be between about 85 degrees and about 115 degrees, between about 76 degrees and about 111 degrees, between about 80 degrees and about 100 degrees, between about 85 degrees and about 95 degrees, etc.

In one or more embodiments, the distal portion 150b of a catheter 18A introduced from the right side of the patient may further include the substantially straight seventh region 700. In alternative embodiments, the catheter 18A does not include the substantially straight seventh region 700. Inclusion or of the substantially straight seventh region 700 may advantageously provide a better anatomical fit for more efficient and accurate placement of the distal end 15b adjacent the LBB for a majority of heart sizes. For example, the substantially straight seventh region 700 may provide an anatomical fit to the tortuous blood vessel pathway through the patient to the heart. The preset curves will allow the catheter 18A to follow the curve of the superior vena cava and reach the target location quickly and without requiring manual steering of the catheter 18A. Inclusion of the substantially straight seventh region 700 may provide a stiffness or hardness which may advantageously provide optimal rigidity and structure during implantation. The inclusion of the substantially straight seventh region 700 of catheter 18A may allow the catheter 18A to be moved smoothly along the vessel when the catheter 18A is advanced to the right atrium. Additionally, the gradual transition from the substantially straight proximal section 99 to the substantially straight seventh region 700 allows for optimized geometry for ease or efficiency of implantation, allows for ease of manufacturing, and also allows for a separate substantially straight seventh region 700 which may have a different stiffness (or other variable, such as length or material) compared to the immediately the surrounding regions on the proximal and distal ends of the substantially straight seventh region 700. The inclusion of substantially straight seventh region 700, the gradual transition, and the stiffness may be optimized for implantation of the catheter 18A in a patient. Omission of the substantially straight seventh region 700 may be beneficial for left side access, as discussed herein.

In some embodiments, the substantially straight seventh region 700 may be distal to the curved sixth region 600. The substantially straight seventh region 700 may be proximal to the first region 100 and/or the curved second region 200. The substantially straight seventh region 700 may define a seventh length L7 lying within the first plane 17. The substantially straight seventh region 700 may be operatively coupled to the curved sixth region 600 such that it extends distally from the curved sixth region 600 along the first plane 17. L7 may be greater than L1, L3, L4, and/or L5. L7 may be less than L6 and/or L2.

In alternative embodiments, the substantially straight seventh region 700 is curved and is not perfectly straight, and the substantially straight seventh region 700 may have a seventh angle A7 (not shown) and a seventh radius R7 (not shown). A7 may be defined as the angle within that same imaginary circle which encompasses the substantially straight seventh region 700. In such embodiments, the seventh angle A7 may be between about 1 degree and about 10 degrees.

The seventh length L7 may be between about 25 mm and about 175 mm. A larger sized catheter 18A introduced from the right side of the patient may include an L7 between about 50 mm and about 150 mm. A smaller sized catheter 18A introduced from the right side of the patient may include an L7 between about 50 mm and about 150 mm. In at least one embodiment, L7 may be about 115 mm. In other embodiments of either sized catheter 18A introduced from the right side of the patient, L7 may be between about 60 mm and about 140 mm, between about 75 mm and about 125 mm, between about 82 mm and about 111 mm, between about 91 mm and about 100 mm, etc.

It can be difficult to advance the lead (e.g., cardiac conduction system pacing lead 18) out of the catheter 18A introduced from the either of the left or right sides of the patient without forces acting on an inner wall of the catheter 18A to push it back from the desired placement. This may result in difficulty in correctly placing the lead. The stiffness and curvilinear structure of the catheter 18A may help offset such difficulties. For example, the curvilinear structure may provide structural stiffness and resistance to torque. Further, for example, the materials used may provide stiffness and resistance to torque.

In one or more embodiments, including the larger and smaller sized catheters 18A introduced from either of the left or right sides of the patient, the proximal portion 99 is stiffer than the distal portion 150b. Stiffness may be defined as the ability of a material to resist elastic deformation when a load is applied. In one or more embodiments, the curved sixth region 600, the substantially straight seventh region 700, the first region 100, and the curved second region 200 are each stiffer than each of the third region 300, the curved fourth region 400, and the fifth region 500. When these relative stiffnesses are realized, the stiffness of the proximal portion 99 provides greater strength to a large section of the elongate tubular member 14 such that there is more resistance against movement of the catheter 18A during advancement of the lead out of the catheter 18A.

More specifically, the curved second region 200 may have a greater stiffness than curved fourth region 400, which may advantageously provide greater stability and rigidity and provide back pressure within the right ventricle on the wall opposite the ventricular septal wall during implantation. Additionally, such stiffness may advantageously provide additional flexibility in the curved fourth region 400 to obtain proper orientation adjacent the septal wall without damaging the septal wall.

Further, the proximal portion 99 may have greater stiffness than the distal portion 150b, which may advantageously provide greater stability and rigidity and provide back pressure within the right ventricle on the wall opposite the ventricular septal wall during implantation. Additionally, such stiffness may advantageously provide additional flexibility in the distal portion 150b to obtain proper orientation adjacent the septal wall without damaging the septal wall.

Further, the curved sixth region 600 may have greater stiffness than the curved second region 200, which may advantageously provide greater stability and rigidity and provide back pressure within the right ventricle on the wall opposite the ventricular septal wall during implantation. Additionally, such stiffness may advantageously provide additional flexibility in the curved second region 200 to obtain proper orientation adjacent the septal wall without damaging the septal wall. In some embodiments, the curved sixth region 600 and the curved second region 200 may have similar stiffness, or may have the same stiffness, which may advantageously provide greater stability and rigidity and provide back pressure as described herein along a greater section of the catheter 18A.

Further, the substantially straight seventh region 700 may have greater stiffness than the curved second region 200, which may advantageously provide greater stability and rigidity and provide back pressure within the right ventricle on the wall opposite the ventricular septal wall during implantation. Additionally, such stiffness may advantageously provide additional flexibility in the curved second region to obtain proper orientation adjacent the septal wall without damaging the septal wall. In some embodiments, the substantially straight seventh region 700 and the curved second region 200 may have similar stiffness, or may have the same stiffness, which may advantageously provide greater stability and rigidity and provide back pressure as described herein along a greater section of the catheter 18A.

Further, the first region 100 and the curved second region 200 may each have greater stiffness than each of the third region 300 and the curved fourth region 400, which may advantageously provide greater stability and rigidity and provide back pressure within the right ventricle on the wall opposite the ventricular septal wall during implantation. Additionally, such stiffness may advantageously provide additional flexibility in the third region 300 and the curved fourth region 400 to obtain proper orientation adjacent the septal wall without damaging the septal wall.

Further, the curved sixth region 600, the substantially straight seventh region 700, the first region 100, and the curved second region 200 may each have greater stiffness than each of the third region 300 and the curved fourth region 400, which may advantageously provide greater stability and rigidity and provide back pressure within the right ventricle on the wall opposite the ventricular septal wall during implantation. Additionally, such stiffness may advantageously provide flexibility in the third region 300 and the curved fourth region 400 to obtain proper orientation adjacent the septal wall without damaging the septal wall.

The catheter 18A may be constructed using one or more materials. For example, the elongate tubular member 14 may be constructed of concentric layers of materials, including a hydrophilic inner liner, an intermediate braid, and an outer elastomeric material (e.g., a polyether block amide material). The outer material may also include a nylon material (e.g., Nylon-12). The outer material may be a combination of elastomeric and nylon materials. The intermediate braid may include a reinforced braid pattern and braid wire such as a braid wire with diamond. The braid wire may be between 0.001 inches and 0.0025 inches. The braid may be a regular braid or a Hercules braid, and may have 8, 16, or 32 wires. A regular braid pattern typically uses a one-under-two, over-two pattern. A diamond braid pattern typically uses a two-under-two, over-two wire pattern. The diamond braid pattern may provide better torque and more stiffness. A Hercules braid pattern typically uses a one-over-three, one-under-three pattern, which may provide even more stiffness. The hydrophilic inner liner may allow the medical device (e.g. cardiac conduction system pacing lead 18) to slide relative to the catheter 18A more easily due to reduced friction. This also results in less force exerted on the inner walls of the catheter 18A, so the catheter 18A does not move out of position while the medical device is being advanced out of the catheter 18A.

In one or more embodiments, each of the various regions of the catheter 18A may have a specific hardness (e.g., as measured using a durometer). Hardness may be defined as the ability of a material to resist puncture. In one or more embodiments, the proximal portion 99 may have a Shore D hardness of about 70 D to about 74 D. In one or more embodiments, the first region 100 and the curved second region 200 may have a Shore D hardness of about 55 D to about 74 D. In one or more embodiments, the third region 300, the curved fourth region 400, and the fifth region 500 may have a Shore D hardness of about 30 D to about 72 D. In one or more embodiments, the curved sixth region 600 and the substantially straight seventh region 700 may have a Shore D hardness of about 55 D to about 74 D.

In one or more embodiments, each of the various regions of the catheter 18A may have a specific stiffness (e.g., as measured using a three point stiffness test). The stiffness of various materials may be defined in terms of a tensile modulus measured in megapascals (MPa). The tensile modulus of various materials may be as follows: the stiffness of Nylon-12 may be about 1400 MPa. The stiffness of Pebax74D may be about 703 MPa. The stiffness of Pebax72D may be about 510 MPa. The stiffness of Pebax70D may be about 414 MPa. The stiffness of Pebax63D may be about 307 MPa. The stiffness of Pebax55D may be about 170 MPa. The stiffness of Pebax45D may be about 88 MPa. The stiffness of Pebax40D may be about 73 MPa. The stiffness of Pebax35D may be about 19 MPa.

In one or more embodiments, the proximal portion 99 may have an outer jacket or layer constructed out of Nylon-12. In one or more embodiments, the first region 100 may have an outer jacket or layer constructed out of Pebax74D. In one or more embodiments, the curved second region 200 may have an outer jacket or layer constructed out of Pebax72D. In one or more embodiments, the third region 300 may have an outer jacket or layer constructed out of Pebax70D. In one or more embodiments, the curved fourth region 400 may have an outer jacket or layer constructed out of Pebax63D. In one or more embodiments, the fifth region 500 may have an outer jacket or layer constructed out of Pebax55D. In one or more embodiments, the curved sixth region 600 may have an outer jacket or layer constructed out of Pebax45D. In one or more embodiments, the substantially straight seventh region 700 may have an outer jacket or layer constructed out of Pebax40D. In alternative embodiments, each of the regions 100-700 may be constructed using one or more of any of the materials discussed herein, which may result in various stiffnesses of each of the regions 100-700.

In alternative embodiments, material with a lower modulus or with a higher modulus may be used. The relative stiffness between regions may remain as described herein. Thus, if the modulus changes, other variables (e.g., amount of material used, or the volume of material) may be changed to retain the relative stiffness between regions as described herein.

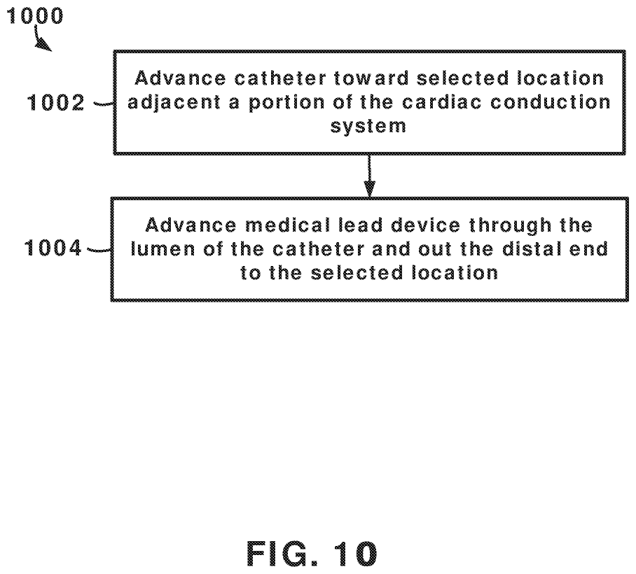

FIG. 10 is a flowchart of one method 1000 of implanting a medical device using the catheter 18A. Although described in regard to cardiac conduction system therapy lead 18 and catheter 18A, the method 1000 can be utilized with any suitable medical device and catheter. The method 1000 may include advancing the catheter 18A toward a selected location adjacent a portion of the cardiac conduction system (e.g., the left bundle branch of a patient via the right ventricle) 1002. The method 1000 may further include advancing the medical device through the lumen 14A and out the distal end 15b of the elongate body to the selected location for at least one of therapy delivery or sensing 1004.

The catheter 18A used in method 1000 may be the same or similar to the catheter 18A discussed herein. The catheter 18A may include a preformed elongate tubular member 14. The preformed elongate tubular member 14 may extend from a proximal end 15a to a distal end 15b. The pre-formed elongate tubular member 14 may define a lumen 14A extending between the proximal end 15a and the distal end 15b. The preformed elongate tubular member 14 may include a proximal portion 99 adjacent the proximal end 15a. The preformed elongate tubular member may include a distal portion 150b adjacent the distal end 15b. The distal portion 150b may define a curvilinear shape in a relaxed state. The curvilinear shape and a stiffness of the catheter 18A may promote advancement of the medical device. Such advancement of the medical device may be without relative movement of the catheter 18A during advancement of the medical device.

Various examples have been described. These and other examples are within the scope of the following claims. For example, a single chamber, dual chamber, or triple chamber pacemakers (e.g., CRT-P) or ICDs (e.g., CRT-D) devices can be used to implement the illustrative methods described herein.

### ILLUSTRATIVE EXAMPLES

While the present disclosure is not so limited, an appreciation of various aspects of the disclosure will be gained through a discussion of the specific illustrative examples provided below. Various modifications of the illustrative examples, as well as additional examples of the disclosure, will become apparent herein.

Example Ex 1: A catheter configured to deliver a lead or other secondary device into a patient's heart and adjacent a portion of the left bundle branch of the patient via the right ventricle, wherein the catheter comprises:
an elongate tubular member comprising a proximal end, a distal end sized for introduction into a patient's body, and a lumen extending therebetween,
wherein the elongate tubular member is pre-formed and further comprises a distal portion adjacent the distal end and a proximal portion adjacent the proximal end, and the distal portion defines a curvilinear shape in a relaxed state,
wherein the distal portion comprises:
a curved second region defining a second radius and a second angle lying within a first plane; and
a curved fourth region distal to the curved second region and defining a fourth radius and a fourth angle lying within a second plane different than the first plane,
wherein the first plane and the second plane intersect at a plane intersect angle between about 50 degrees to about 110 degrees,
wherein the second radius is about 25 mm to about 115 mm,
wherein the fourth radius is about 10 mm to about 35 mm, and
wherein the second radius is greater than the fourth radius.

Example Ex2: The catheter of Example Ex1, wherein the distal portion further comprises:
a first region proximal to the curved second region and defining a first length lying within the first plane.

Example Ex3: The catheter of any one of Examples Ex1-2, wherein the distal portion further comprises:
a third region distal to the curved second region and defining a third length lying within the first plane.

Example Ex4: The catheter of any one of Examples Ex1-3,
wherein the second angle is about 85 degrees to about 230 degrees, and
wherein the fourth angle is about 55 degrees to about 180 degrees.

Example Ex5: The catheter of any one of Examples Ex2-4,
wherein the proximal portion is stiffer than the distal portion, and
wherein the first region and the curved second region are stiffer than the third region and the curved fourth region.

Example Ex6: The catheter of any one of Examples Ex1-5, wherein the distal portion further comprises:
a fifth region distal to the curved fourth region and proximal to the distal end and defining a fifth length lying within the second plane, wherein the fifth length is less than or equal to about 5 mm.

Example Ex7: The catheter of any one of Examples Ex1-6, wherein the catheter is configured to be implanted from at least one of the left cephalic vein, the axillary vein, and the left subclavian vein of the patient through the right atrium and into the right ventricle of the patient's heart.

Example Ex8: The catheter of any one of Examples Ex3-6, where the distal portion further comprises:
a curved sixth region defining a sixth radius and a sixth angle lying within the first plane proximal to the first region; and
a substantially straight seventh region distal to the curved sixth region and proximal to the first region and defining a seventh length lying within the first plane,
wherein the sixth angle is about 80 degrees to about 120 degrees,
wherein the proximal portion is stiffer than the distal portion, and
wherein the curved sixth region, the substantially straight seventh region, the first region and the curved second region are stiffer than the third region and the curved fourth region.

Example Ex9: The catheter of Example Ex8, wherein the catheter is configured to be implanted from at least one of the right cephalic vein, the right subclavian vein, and the axillary vein of the patient through the right atrium and into the right ventricle of the patient's heart.

Example Ex10: The catheter of any one of Examples Ex2-9, wherein the first region further defines a first radius and a first angle, and wherein the first radius is about 80 mm to about 120 mm and wherein the first angle is less than or equal to about 60 degrees.

Example Ex11: The catheter of any one of Examples Ex3-10, wherein the third length is about 1 mm to about 10 mm.

Example Ex12: The catheter of any one of Examples Ex7-11, wherein the sixth radius is about 40 mm to about 80 mm.

Example Ex13: The catheter of any one of Examples Ex7-11, wherein the seventh length is about 50 mm to about 150 mm.

Example Ex14: The catheter of any one of Examples Ex2-13, wherein the first region defines a first length and the curved second region defines a second length, and wherein the second length is greater than the first length.

Example Ex15: The catheter of Example Ex14, wherein the curved fourth region defines a fourth length, and wherein the fourth length is greater than the first length, and wherein the fourth length is less than the second length.

Example Ex16: The catheter of any one of Examples Ex7-15, wherein the curved sixth region defines a sixth length, and wherein the sixth length is greater than the second length.

Example Ex17: The catheter of any one of Examples Ex1-16, wherein the proximal portion is at least partially composed of a material that has a shore hardness of about 70 D to about 74 D and additionally or alternatively may have a Young's modulus of about 1400 MPa.

Example Ex18: The catheter of any one of Examples Ex2-17, wherein the first region and the curved second region are each at least partially composed of a material that has a shore hardness of about 55 D to about 74 D and additionally or alternatively may have a Young's Modulus of about 703 MPa to about 510 MPa.

Example Ex19: The catheter of any one of Examples Ex6-18, wherein the third region, the curved fourth region, and the fifth region are each at least partially composed of a material that has a shore hardness of about 30 D to about 72 D and additionally or alternatively may have a Young's Modulus of about 414 MPa to about 170 MPa.

Example Ex20: The catheter of any one of Examples Ex7-19, wherein the curved sixth region and the substantially straight seventh region are each at least partially composed of a material that has a shore hardness of about 55 D to about 74 D and additionally or alternatively may have a Young's Modulus of about 88 MPa to about 19 MPa.

Example Ex21: The catheter of any one of Examples Ex1-20, wherein when the first plane is oriented substantially parallel to the coronal plane of the body, the fourth curved region extends in a direction from the coronal plane towards a dorsal section of the body.

Example Ex22: a method comprising:
advancing a catheter toward a selected location adjacent a portion of the left bundle branch of a patient via the right ventricle, wherein the catheter comprises:
   a pre-formed elongate tubular member extending from a proximal end to a distal end and defining a lumen extending therebetween, the elongate tubular member comprising a proximal portion adjacent the proximal end and a distal portion adjacent the distal end, wherein the distal portion defines a curvilinear shape in a relaxed state, and
wherein the distal portion comprises:
   a curved second region defining a second radius and a second angle lying within a first plane; and
   a curved fourth region distal to the curved second region and defining a fourth radius and a fourth angle lying within a second plane different than the first plane,
   wherein the first plane and the second plane intersect at a plane intersect angle between about 50 degrees to about 110 degrees,
   wherein the second radius is about 25 mm to about 115 mm,
   wherein the fourth radius is about 10 mm to about 35 mm, and
   wherein the second radius is greater than the fourth radius; and
advancing a medical device through the lumen and out the distal end of the pre-formed elongate tubular member to the selected location for at least one of therapy delivery or sensing,
   wherein the curvilinear shape and a stiffness of the catheter promote advancement of the medical device without relative movement of the catheter during advancement of the medical device.

Example Ex23: A catheter configured to deliver a lead or other secondary device into a patient's heart and adjacent a portion of the left bundle branch of the patient via the right ventricle, wherein the catheter comprises:
an elongate tubular member comprising a proximal end, a distal end sized for introduction into a patient's body, and a lumen extending therebetween,
wherein the elongate tubular member is pre-formed and further comprises a distal portion adjacent the distal end and a proximal portion adjacent the proximal end, and the distal portion defines a curvilinear shape in a relaxed state,
wherein the distal portion comprises:
   a first region distal to the proximal portion and defining a first length lying within the first plane;
   a curved second region distal to the first region and defining a second radius and a second angle lying within a first plane;
   a third region distal to the curved second region and defining a third length lying within the first plane;
   a curved fourth region distal to the curved second region and defining a fourth radius and a fourth angle lying within a second plane different than the first plane;
   a curved sixth region proximal to the first region and defining a sixth radius and a sixth angle lying within the first plane; and
   a substantially straight seventh region distal to the curved sixth region and proximal to the first region and defining a seventh length lying within the first plane;
wherein the first plane and the second plane intersect at a plane intersect angle between about 50 degrees to about 110 degrees,
wherein the second radius is about 25 mm to about 115 mm,
wherein the fourth radius is about 10 mm to about 35 mm,
wherein the sixth radius is about 40 mm to about 80 mm, and
wherein the second radius and the sixth radius are each greater than the fourth radius.

Example Ex24: A catheter configured to deliver a lead or other secondary device into a patient's heart and adjacent a portion of the left bundle branch of the patient via at least one of the left cephalic vein, the axillary vein, and the left subclavian vein of the patient through the right atrium and into the right ventricle of the patient's heart, wherein the catheter comprises:
an elongate tubular member comprising a proximal end, a distal end sized for introduction into a patient's body, and a lumen extending therebetween,
wherein the elongate tubular member is pre-formed and further comprises a distal portion adjacent the distal end and a proximal portion adjacent the proximal end, and the distal portion defines a curvilinear shape in a relaxed state,
wherein the distal portion comprises:
   a first region defining a first radius and a first angle lying within a first plane;
   a curved second region distal to the first region and defining a second radius and a second angle lying within the first plane;
   a third region distal to the curved second region and defining a third length lying within the first plane; and
   a curved fourth region distal to the third region and defining a fourth radius and a fourth angle lying within a second plane different than the first plane,
wherein the first plane and the second plane intersect at a plane intersect angle between about 50 degrees to about 110 degrees.

Example Ex25: A catheter configured to deliver a lead or other secondary device into a patient's heart and adjacent a portion of the left bundle branch of the patient via at least one of the right cephalic vein, the right subclavian vein, and the axillary vein of the patient through the right atrium and into the right ventricle of the patient's heart, wherein the catheter comprises:
an elongate tubular member comprising a proximal end, a distal end sized for introduction into a patient's body, and a lumen extending therebetween,
wherein the elongate tubular member is pre-formed and further comprises a distal portion adjacent the distal end and a proximal portion adjacent the proximal end, and the distal portion defines a curvilinear shape in a relaxed state,
wherein the distal portion comprises:
   a first region defining a first radius and a first angle lying within a first plane;
   a curved second region distal to the first region and defining a second radius and a second angle lying within the first plane;
   a third region distal to the curved second region and defining a third length lying within the first plane;
   a curved fourth region distal to the third region and defining a fourth radius and a fourth angle lying within a second plane different than the first plane;
   a fifth region distal to the curved fourth region and proximal to the distal end and defining a fifth length lying within the second plane;
   a curved sixth region distal to the proximal portion and defining a sixth radius and a sixth angle, lying within the first plane proximal to the first region; and
   a substantially straight seventh region distal to the curved sixth region and proximal to the first region and defining a seventh length lying within the first plane,
wherein the first plane and the second plane intersect at a plane intersect angle between about 50 degrees to about 110 degrees.

This disclosure has been provided with reference to illustrative embodiments and examples and is not meant to be construed in a limiting sense. As described previously, one skilled in the art will recognize that other various illustrative applications may use the techniques as described herein to take advantage of the beneficial characteristics of the devices and methods described herein. Various modifications of the illustrative embodiments and examples will be apparent upon reference to this description.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g., 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a mobile user device may be operatively coupled to a cellular network transmit data to or receive data therefrom).

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

1. A catheter configured to deliver a lead or other secondary device into a patient's heart and adjacent a portion of the left bundle branch of the patient via the right ventricle, wherein the catheter comprises:
an elongate tubular member comprising a proximal end, a distal end sized for introduction into a patient's body, and a lumen extending therebetween,
wherein the elongate tubular member is pre-formed and further comprises a distal portion adjacent the distal end and a proximal portion adjacent the proximal end, and the distal portion defines a curvilinear shape in a relaxed state,
wherein the distal portion comprises:
a curved second region defining a second radius and a second angle lying within a first plane; and
a curved fourth region distal to the curved second region and defining a fourth radius and a fourth angle lying within a second plane different than the first plane,
wherein the first plane and the second plane intersect at a plane intersect angle between about 50 degrees to about 110 degrees,
wherein the second radius is about 25 mm to about 115 mm,
wherein the fourth radius is about 10 mm to about 35 mm, and
wherein the second radius is greater than the fourth radius.

2. The catheter of claim 1, wherein the distal portion further comprises:
a first region proximal to the curved second region and defining a first length lying within the first plane.

3. The catheter of claim 1, wherein the distal portion further comprises:
a third region distal to the curved second region and defining a third length lying within the first plane.

4. The catheter of claim 1,
wherein the second angle is about 85 degrees to about 230 degrees, and
wherein the fourth angle is about 55 degrees to about 180 degrees.

5. The catheter of claim 3,
wherein the proximal portion is stiffer than the distal portion, and
wherein the first region and the curved second region are stiffer than the third region and the curved fourth region.

6. The catheter of claim 1, wherein the distal portion further comprises:
a fifth region distal to the curved fourth region and proximal to the distal end and defining a fifth length lying within the second plane, wherein the fifth length is less than or equal to about 5 mm.

7. The catheter of claim 1, wherein the catheter is configured to be implanted from at least one of the left cephalic vein, the axillary vein, and the left subclavian vein of the patient through the right atrium and into the right ventricle of the patient's heart.

8. The catheter of claim 3, where the distal portion further comprises:
a curved sixth region defining a sixth radius and a sixth angle lying within the first plane proximal to the first region; and
a substantially straight seventh region distal to the curved sixth region and proximal to the first region and defining a seventh length lying within the first plane,
wherein the sixth angle is about 80 degrees to about 120 degrees,
wherein the proximal portion is stiffer than the distal portion, and
wherein the curved sixth region, the substantially straight seventh region, the first region and the curved second region are stiffer than the third region and the curved fourth region.

9. The catheter of claim 8, wherein the catheter is configured to be implanted from at least one of the right cephalic vein, the right subclavian vein, and the axillary vein of the patient through the right atrium and into the right ventricle of the patient's heart.

10. The catheter of claim 2, wherein the first region further defines a first radius and a first angle, and wherein the first radius is about 80 mm to about 120 mm and wherein the first angle is less than or equal to about 60 degrees.

11. The catheter of claim 3, wherein the third length is about 1 mm to about 10 mm.

12. The catheter of claim 7, wherein the sixth radius is about 40 mm to about 80 mm.

13. The catheter of claim 7, wherein the seventh length is about 50 mm to about 150 mm.

14. The catheter of claim 2, wherein the first region defines a first length and the curved second region defines a second length, and wherein the second length is greater than the first length.

15. The catheter of claim 1, wherein the proximal portion is at least partially composed of a material that has a shore hardness of about 70 D to about 74 D.

16. The catheter of claim 2, wherein the first region and the curved second region are each at least partially composed of a material that has a shore hardness of about 55 D to about 74 D.

17. The catheter of claim 6, wherein the third region, the curved fourth region, and the fifth region are each at least partially composed of a material that has a shore hardness of about 30 D to about 72 D.

18. The catheter of claim 7, wherein the curved sixth region and the substantially straight seventh region are each at least partially composed of a material that has a shore hardness of about 55 D to about 74 D.

19. (canceled)

20. A method comprising:
advancing a catheter toward a selected location adjacent a portion of the left bundle branch of a patient via the right ventricle, wherein the catheter comprises:
a pre-formed elongate tubular member extending from a proximal end to a distal end and defining a lumen extending therebetween, the elongate tubular member comprising a proximal portion adjacent the proximal end and a distal portion adjacent the distal end, wherein the distal portion defines a curvilinear shape in a relaxed state, and
wherein the distal portion comprises:
a curved second region defining a second radius and a second angle lying within a first plane; and
a curved fourth region distal to the curved second region and defining a fourth radius and a fourth angle lying within a second plane different than the first plane,
wherein the first plane and the second plane intersect at a plane intersect angle between about 50 degrees to about 110 degrees,
wherein the second radius is about 25 mm to about 115 mm,
wherein the fourth radius is about 10 mm to about 35 mm, and
wherein the second radius is greater than the fourth radius; and
advancing a medical device through the lumen and out the distal end of the pre-formed elongate tubular member to the selected location for at least one of therapy delivery or sensing,
wherein the curvilinear shape and a stiffness of the catheter promote advancement of the medical device without relative movement of the catheter during advancement of the medical device.

21. A catheter configured to deliver a lead or other secondary device into a patient's heart and adjacent a portion of the left bundle branch of the patient via the right ventricle, wherein the catheter comprises:
an elongate tubular member comprising a proximal end, a distal end sized for introduction into a patient's body, and a lumen extending therebetween,
wherein the elongate tubular member is pre-formed and further comprises a distal portion adjacent the distal end and a proximal portion adjacent the proximal end, and the distal portion defines a curvilinear shape in a relaxed state,
wherein the distal portion comprises:
a first region distal to the proximal portion and defining a first length lying within the first plane;
a curved second region distal to the first region and defining a second radius and a second angle lying within a first plane;
a third region distal to the curved second region and defining a third length lying within the first plane;
a curved fourth region distal to the curved second region and defining a fourth radius and a fourth angle lying within a second plane different than the first plane;
a curved sixth region proximal to the first region and defining a sixth radius and a sixth angle lying within the first plane; and
a substantially straight seventh region distal to the curved sixth region and proximal to the first region and defining a seventh length lying within the first plane;
wherein the first plane and the second plane intersect at a plane intersect angle between about 50 degrees to about 110 degrees,
wherein the second radius is about 25 mm to about 115 mm,
wherein the fourth radius is about 10 mm to about 35 mm,
wherein the sixth radius is about 40 mm to about 80 mm, and
wherein the second radius and the sixth radius are each greater than the fourth radius.
